(19) United States
(12) Patent Application Publication
(10) Pub. No.: US 2021/0191889 A1
(43) Pub. Date: Jun. 24, 2021

(54) A LABORATORY EQUIPMENT INTERFACE AND A METHOD OF ASSOCIATING EMPIRICAL INFORMATION FOR A LABORATORY ANIMAL FROM A PLURALITY OF ANIMAL LABORATORY DEVICES WITHIN A DATA STOREDEVICES WITHIN A DATA STORE

(71) Applicant: SOMARK GROUP LIMITED, SYDNEY, New South Wales (AU)

(72) Inventors: Adrian Knight, SYDNEY, New South Wales (AU); Paul Donohoe, SYDNEY, New South Wales (AU)

(73) Assignee: Somark Group Ltd, SYDNEY, New South Wales (AU)

(21) Appl. No.: 16/755,309

(22) PCT Filed: Oct. 12, 2018
(86) PCT No.: PCT/AU2018/051103

§ 371 Date: Apr. 10, 2020

Related U.S. Application Data
(60) Provisional application No. 62/571,769, filed on Oct. 12, 2017.

(51) Int. Cl. G06F 13/20; G08B 21/18; H04Q 9/00

(52) U.S. Cl. CPC G06F 13/20; H04Q 9/00; G08B 21/18

(57) ABSTRACT

Disclosed herein is a laboratory equipment interface comprising at least one communication interface for receiving information from a plurality of animal laboratory device. The laboratory equipment interface comprises a communications interface for communicating with a remote data store. The laboratory equipment interface comprises a processor). The laboratory equipment interface comprises tangible media including program instructions which when executed by the processor causes the processor to perform the steps of an embodiment of a method.

### TECHNICAL FIELD

The disclosure herein generally relates to a laboratory equipment interface and a method of associating empirical information for a laboratory animal from a plurality of animal laboratory devices within a data store.

### BACKGROUND

An animal laboratory may have many pieces of laboratory equipment used to generate empirical information about experimental animal subjects. Some animal laboratories have thousands or millions of animals in the form of rodents, for example mice and/or rates. It is laborious to generate empirical information for large animal populations, especially when using many different pieces of laboratory equipment and manually entering the empirical data against a record for each animal.

Mistakes do occur in recording empirical values. An empirical value may be recorded against the wrong animal identity. This may effect the integrity of the experimental data and the experiment itself. Not only may this mask the outcomes of the experiment, it may be extremely costly for experiments involving a large number of animals and/or expensive animals. Some mice cost $1,000 or more.

### SUMMARY

Disclosed herein is a laboratory equipment interface. The laboratory equipment interface comprises at least one communication interface for receiving information from a plurality of animal laboratory devices. The laboratory equipment interface comprises a communications interface for communicating with a remote data store. The laboratory equipment interface comprises a processor configured to generate an information transmission unit comprising empirical information relating to a laboratory animal received from one of the plurality of animal laboratory device and animal identification information indicative of the identity of the animal, and configured to transmit the information transmission unit to the remote data store via the communication interface for association of the empirical information with the animal identification information within the remote data store.

In an embodiment, the processor is configured to receive the animal identification information from one of the plurality of animal laboratory devices configured to read an identification tag. Alternatively or additionally, the animal identification information is received from the remote data store.

In an embodiment, the processor is configured to receive user instruction information indicative of an instruction for a user to initiate the generation of the empirical information relating to the laboratory animal and communicate to the user the instruction. The processor may be configured to subsequently confirm that the user instruction information comprises animal identification information indicative of the animal.

In an embodiment, the processor is configured to trigger an alert when the processor determines that the empirical information satisfies an alert condition.

An embodiment comprises pre-loaded animal laboratory device software for each of the plurality of animal laboratory devices.

In an embodiment, the information transmission unit comprises a file

An embodiment has a network address and the processor is configured to include the network address in the information transmission unit.

In an embodiment, the at least one communication interface comprises at least one personal area network interface.

In an embodiment, the communications interface comprises a local area network interface.

Disclosed herein is a laboratory equipment interface. The laboratory equipment interface comprises at least one communication interface for receiving information from an animal laboratory device. The laboratory equipment interface comprises a communications interface for communicating with a remote data store. The laboratory equipment interface comprises a processor and tangible media including program instructions which when executed by the processor causes the processor to generate an information transmission unit comprising empirical information relating to a laboratory animal received from one of the plurality of animal laboratory devices and animal identification information indicative of the identity of the animal, and transmit the information transmission unit to the remote data store via the communication interface for association of the empirical information with the animal identification information within the remote data store.

Disclosed herein is a method of associating within a data store empirical information for a laboratory animal from a plurality of animal laboratory devices. The method comprises the step of receiving empirical information for a laboratory animal from each of a plurality of animal laboratory devices via at least one communications interface. The method comprises the step of generating an information transmission unit for each of the plurality of animal laboratory devices, the information transmission unit comprising the empirical information received therefrom and animal identification information indicative of the identity of the animal. The method comprises the step of sending, via a communications interface for communication with a remote data store, the information transmission unit generated for each of the plurality of connected animal laboratory devices to the remote data store for association of the empirical information with the animal identity information within the remote data store.

An embodiment comprises the step of receiving animal identification information. The animal identification information may be received from one of the plurality of animal laboratory devices configured to read an identification tag attached to the animal. Alternatively or additionally, the animal identification information is received from the data store.

An embodiment comprises the step of receiving user instruction information indicating that a user should initiate the generation of the empirical information relating to the laboratory animal and communicate to the user the user instruction information, and subsequently confirming that the user instruction information comprises animal identification information indicative of the animal.

An embodiment comprises the step of triggering an alert when the processor determines that the empirical information satisfies an alert condition.

An embodiment comprises the step of preloading animal laboratory device software for each of the plurality of animal laboratory devices.

In an embodiment, the information transmission unit comprises a file.

An embodiment comprises the step of including a network address of a laboratory equipment interface that performs the steps of a method disclosed above.

Disclosed herein is a laboratory equipment interface comprising a processor and tangible media including program instructions which when executed by the processor causes the processor to perform a method disclosed above.

Disclosed herein is non-transitory processor readable tangible media including program instructions which when executed by a processor causes the processor to perform a method disclosed above.

Disclosed herein is a computer program for instructing a processor, which when executed by the processor causes the processor to perform a method disclosed above.

Any of the various features of each of the above disclosures, and of the various features of the embodiments described below, can be combined as suitable and desired.

### BRIEF DESCRIPTION OF THE FIGURES

Embodiments will now be described by way of example only with reference to the accompanying figures in which:

FIG. 1 shows a block diagram of an embodiment of a laboratory equipment interface.

FIG. 2 shows a flow chart of an embodiment of a method that can be performed using the laboratory equipment interface of FIG. 1.

FIG. 3 shows an example of a user interface on a electronic display of the laboratory equipment interface of FIG. 1.

### DESCRIPTION OF EMBODIMENTS

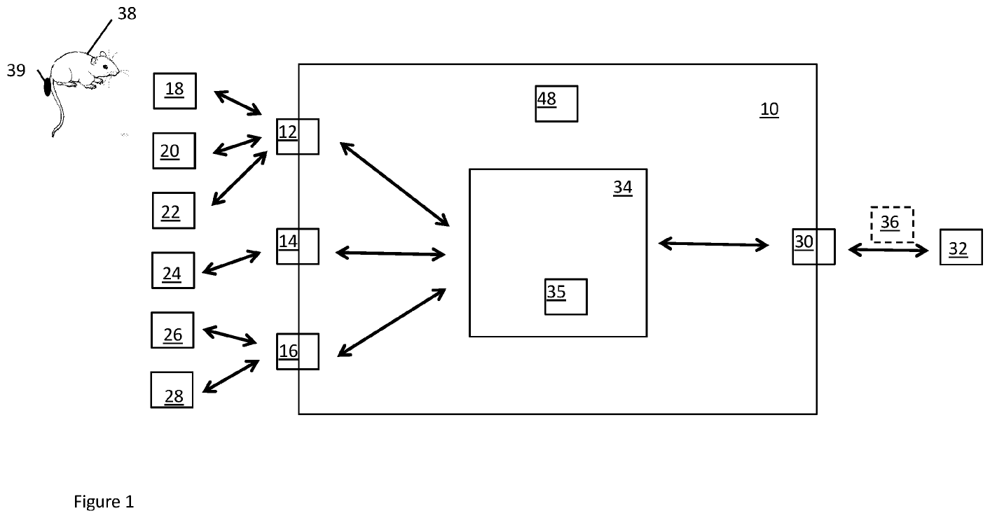

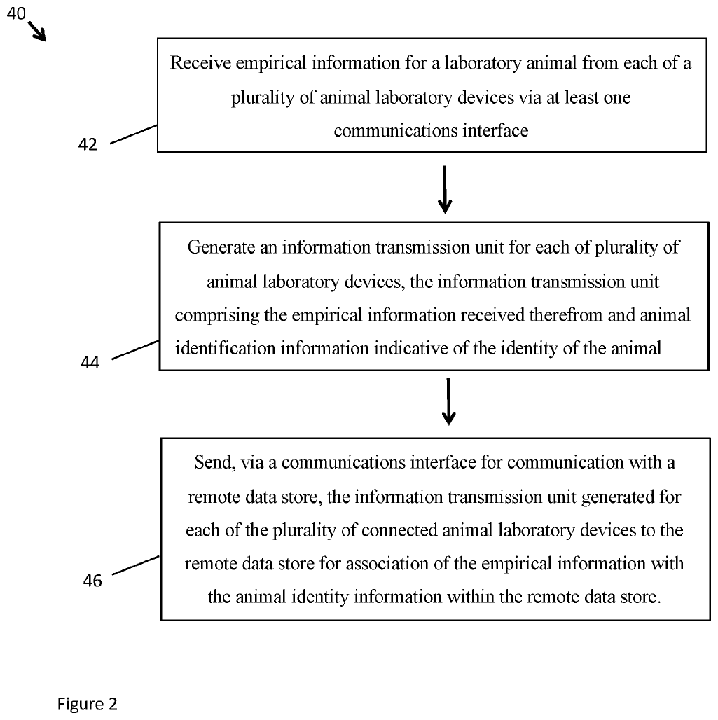

FIG. 1 shows a block diagram of an embodiment of a laboratory equipment interface generally indicated by the numeral 10. The laboratory equipment interface 10 comprises at least one communication interface 12-16 for receiving information from a plurality of animal laboratory device 18-28. The laboratory equipment interface 10 comprises a communications interface 30 for communicating with a remote data store 32. The laboratory equipment interface 10 comprises a processor 34. The laboratory equipment interface comprises tangible media 35 including program instructions which when executed by the processor 34 causes the processor 34 to perform the steps 42-44 of an embodiment of a method, a flow chart for which is shown in FIG. 2. The processor 34 generates an information transmission unit 36 comprising empirical information relating to a laboratory animal 38 received from at least one of the plurality of animal laboratory devices 18-28 and animal identification information indicative of the identity of the animal 38. The processor 34 transmits the information transmission unit 36 to the remote data store 32 via the communication interface 30 for association of the empirical information with the animal identification information within the remote data store 32. Steps that can be combined as suitable and desired with the above steps are described below with reference to the laboratory equipment interface 10. The animal is in the form of a mouse or rat, but may be any suitable animal.

The processor 34 receives animal identification information by animal laboratory device 18 reading an identification tag 39, which in this embodiment is attached to the animal. In this embodiment, the identification tag 39 is a radio frequency identification tag implanted in the animal, however it may be an identification tag readable using machine vision, or generally any suitable form of identification tag. The radio frequency identification tag has stored therein the animal identification information in the form of a sequence of symbols. Multiple devices 18-28 can be simultaneously connected. In an otherwise identical embodiment, a tag identification (TID) is read and sent to the laboratory equipment interface 10, which sends a request and subsequently receives the animal identification information associated with the TID from the data store. The TID and the animal identity are preassociated in the data store. Because the TID and the animal identify information are both indicative of the one animal, the TID is merely a different form of the animal identity information.

An example of the text in an information transmission unit 36 in JSON format is:

```
{
  “studyId”: “study-a”,
  “studyPhaseId”: “Weight gain”,
  “studyExperimentId”: “lb129cfa-c407-4422-b5e9-61faba854e66”,
  “experimentTestId”: null,
  “animalId”: “8bbef86f-373a-446e-89e5-9caeff2ab90a”,
  “type”: “Observational”,
  “startTime”: “2017-07-20T10:54:19.7291261+09:30”,
  “endTime”: “2017-07-20T10:54:19.7291261+09:30”,
  “testMethod”: {
    “type”: “Animal”,
    “animalSpecies”: {
      “family”: “Rodent”,
      “code”: “Mouse”,
      “defaultDescription”: “Mouse”,
      “localizedValues”: [ ],
      “description”: “Mouse”,
      “id”: “Mouse”
    },
    “unitCategory”: {
      “units”: [
        {
          “labels”: [
            “ng”
          ],
          “isBaseUnit”: false,
          “code”: “ng”,
          “defaultDescription”: “ng”,
          “localizedValues”: [ ],
          “description”: “ng”,
          “id”: “cc54de72-24a5-4eb5-8cb8-074a921edc47”
        },
        {
          “labels”: [
            “μg”,
            “μg”,
            “mcg”
          ],
          “isBaseUnit”: false,
          “code”: “ug”,
          “defaultDescription”: “ug”,
          “localizedValues”: [ ],
          “description”: “ug”,
          “id”: “66430fa4-698e-4a77-848d-78db1b872644”
        },
        {
          “labels”: [
            “mg”
          ],
          “isBaseUnit”: false,
          “code”: “mg”,
          “defaultDescription”: “mg”,
          “localizedValues”: [ ],
          “description”: “mg”,
          “id”: “486bfe0a-1004-4012-9e09-15fa8d5b0812”
        },
        {
          “labels”: [
            “g”
          ],
          “isBaseUnit”: true,
          “code”: “g”,
          “defaultDescription”: “g”,
          “localizedValues”: [ ],
          “description”: “g”,
          “id”: “21b4cc43-7854-4273-96ec-284f822532d7”
        },
        {
          “labels”: [
            “kg”
          ],
          “isBaseUnit”: false,
          “code”: “kg”,
          “defaultDescription”: “kg”,
          “localizedValues”: [ ],
          “description”: “kg”,
          “id”: “f144effd-8c4d-4d3f-a127-c1d3b64c2d4d”
        },
        {
          “labels”: [
            “oz”
          ],
          “isBaseUnit”: false,
          “code”: “oz”,
          “defaultDescription”: “oz”,
          “localizedValues”: [ ],
          “description”: “oz”,
          “id”: “751d0cce-0725-4970-ac72-e99d5d15a6ec”
        },
        {
          “labels”: [
            “lb”
          ],
          “isBaseUnit”: false,
          “code”: “lb”,
          “defaultDescription”: “lb”,
          “localizedValues”: [ ],
          “description”: “lb”,
          “id”: “6c1ef3e2-51b6-4665-9ae4-202e27e1c21d”
        },
        {
          “labels”: [
            “st”
          ],
          “isBaseUnit”: false,
          “code”: “st”,
          “defaultDescription”: “st”,
          “localizedValues”: [ ],
          “description”: “st”,
          “id”: “1d000215-9ee4-44e6-8002-b5932fd164c5”
        }
      ],
      “code”: “Mass”,
      “defaultDescription”: “Mass”,
      “localizedValues”: [ ],
      “description”: “Mass”,
      “id”: “Mass”
    },
    “devices”: [
      {
        “code”: “Keyboard”,
        “defaultDescription”: “Keyboard”,
        “localizedValues”: [ ],
        “description”: “Keyboard”,
        “id”: “Device-Keyboard”
      },
      {
        “code”: “Balance”,
        “defaultDescription”: “Balance”,
        “localizedValues”: [ ],
        “description”: “Balance”,
        “id”: “Device-Balance”
      }
    ],
    “title”: “Weigh mouse”,
    “description”: null,
    “bodyPart”: “Whole body”,
    “documentType”: 6,
    “animalModel”: {
      “dorsum”: “animal-models/mouse/not-defined/whole-
body/dorsum.png”,
      “ventrum”: “animal-models/mouse/not-defined/whole-
body/ventrum.png”,
      “left”: “animal-models/mouse/not-defined/whole-body/left.png”,
      “right”: “animal-models/mouse/not-defined/whole-body/right.png”
    },
    “_etag”: “\“00003c00-0000-0000-0000-596ed8b40000\””,
    “id”: “776A5910-4EA0-4625-8D62-AF3123B842A3”
  },
  “results”: [
    {
      “status”: “Active”,
      “replacedResultId”: null,
      “reasons”: null,
      “creator”: {
        “id”: “mburess@somarkinnovations.com”,
        “email”: “mburgess@somarkinnovations.com”,
        “firstName”: “Mike”,
        “lastName”: “Burgess”,
        “title”: “Mr”,
        “jobTitle”: “Pretending”,
        “mobile”: “0430440210”,
        “workPhone”: “0812345678”,
        “active”: true,
        “roles”: [
          “Administrator”,
          “User”
        ],
        “documentType”: 3,
        “userName”: “Mike Burgess”,
        “_etag”: “\“00004400-0000-0000-0000-596ed8b40000\””
      },
      “clientType”: “DataHub”,
      “timestamp”: “2017-07-20T10:54:19.7291261+09:30”,
      “animalSnapshot”: {
        “locationId”: “51f49af9-bef4-466d-88a6-4b8f9452ccd6”,
        “species”: “Mouse”,
        “purpose”: “Experimental”,
        “gender”: “Male”,
        “dateOfBirth”: “2017-03-27T00:00:00”,
        “dateOfDeath”: null,
        “reasonForDeath”: null,
        “lifeStatus”: “Alive”,
        “healthStatus”: null,
        “strain”: “Strain A”,
        “line”: “Line A”,
        “passageNumber”: “P1”,
        “geneticStatus”: “NonGM”,
        “genotype”: “AAAA/aaaa”,
        “phenotype”: “abc-phenotype-1000”,
        “zygosity”: “Homozygous”,
        “notes”: “Cras a dui non mauris eleifend auctor sed quis
ante.”,
        “studyId”: “study-a”,
        “studyExperimentId”: “1b129cfa-c407-4422-b5e9-61faba854e66”,
        “supplierName”: “Primary source”,
        “identity”: {
          “rfIdValue”: “E280117020000184574508E2”,
          “rfIdDate”: “2017-05-10T00:00:00”,
          “tattooIdValue”: “AAA999”,
          “tattooIdDate”: “2017-05-11T00:00:00”,
          “earNotchIdValue”: 10,
          “earNotchIdDate”: “2017-05-13T00:00:00”,
          “earTagIdValue”: “104”,
          “earTagIdDate”: “2017-05-12T00:00:00”,
          “toeClipIdValue”: 890,
          “toeClipIdDate”: “2017-05-15T00:00:00”,
          “supplierIdValue”: “AAAA-GGGG-569b”,
          “supplierIdDate”: “2017-05-16T00:00:00”,
          “visualIdMethod”: “EarNotch”,
          “VisibleId”: “10”
        },
        “documentType”: 4,
        “_etag”: “\“00002b00-0000-0000-0000-596ed8b20000\””,
        “Id”: “8bbef86f-373a-446e-89e5-9caeff2ab90a”
      },
      “locationSnapshot”: “Bubble 2”,
      “baseValue”: {
        “unit”: “g”,
        “value”: 27.782532685798173,
        “resultType”: “Double”
      },
      “recordedValue”: {
        “unit”: “lb”,
        “value”: 0.06125,
        “resultType”: “Double”
      },
      “acquisitionDeviceProperties”: {
        “vendorId”: “067B”,
        “productId”: “2303”,
        “hardwareDeviceId”: “USB\\VID_067B&PID_2303\\6&272C1C87&0&4”,
        “manufacturer”: “Mettler Toledo”,
        “serialNumber”: “B649390484”,
        “model”: “MS3002TS/00”,
        “softwareVersion”: “3.41 (48.28.3.3738.1650)”
      },
      “clientSystemProperties”: {
        “clientType”: 0,
        “hostName”: “DESKTOP-BNT8D6O”,
        “softwareVersion”: “1.0.13.0”
      },
      “id”: “c868e91d-000d-49c8-b318-449102587f68”
    }
  ],
  “resultCount”: 1,
  “documentType”: 14,
  “assignedTo”: “mburgess@somarkinnovations.com”,
  “_etag”: “\“00005d00-0000-0000-0000-597006430000\””,
  “id”: “76398564-50e0-4ca3-9e48-0d61edd8cb82”,
  “_rid”: “rMMGALssKgNVAAAAAAAAAA==”,
  “_self”:
“dbs/rMMGAA==/colls/rMMGALssKgM=/docs/rMMGALssKgNVAAAAAAAAAA==/”,
  “_attachments”: “attachments/”,
  “_ts”: 1500513859
}
```

The animal identification information is used as a key for relating data base records in the data store. This generally eliminates the possibility of human bias and error in experiments. The integrity of experimental studies may be improved. An incorrectly identified and/or processed animal may be euthanised.

When the identification tag is read and received by the laboratory equipment interface 10, the laboratory equipment interface may send the tag ID to the remote data store. The remote data store may respond by sending information back to the laboratory equipment interface 10 in the form of a text file, which in this embodiment is JSON formatted. An example of such information is:

```
{ {
  “animalId”:“8bbef86f-373a-446e-89e5-9caeff2ab90a”,
  “testMethodId”:“776A5910-4EA0-4625-8D62-AF3123B842A3”,
  “type”: “Observational”,
  “locationSnapshot”:“Bubble 2”,
  “acquisitionDeviceProperties”:
  {
      “vendorId”:“067B”,
      “productId”:“2303”,
    “hardwareDeviceId”:“USB\\VID_067B&PID_2303\\6&272C1C87&0&4”,
        “manufacturer”: “Mettler Toledo”,
        “serialNumber”:“B649390484”,
        “model”:“MS3002TS/00”,
        “softwareVersion”:“3.41 (48.28.3.3736.1650)”},
        “recordedValue”:
{
          “unit”: “lb”,
          “value”:0.06125,
          “resultType”:“Double”
        },
        “clientSystemProperties”:
        {
          “clientType”:0,
          “hostName”:“DESKTOP-BNT8D6O”,
          “softwareVersion”:“1.0.13.0”
        },
        “studyId”:“study-a”,
        “studyPhaseId”:“Weight gain”,
        “experimentId”:“lb129cfa-c407-4422-b5e9-61faba854e66”
} }
```

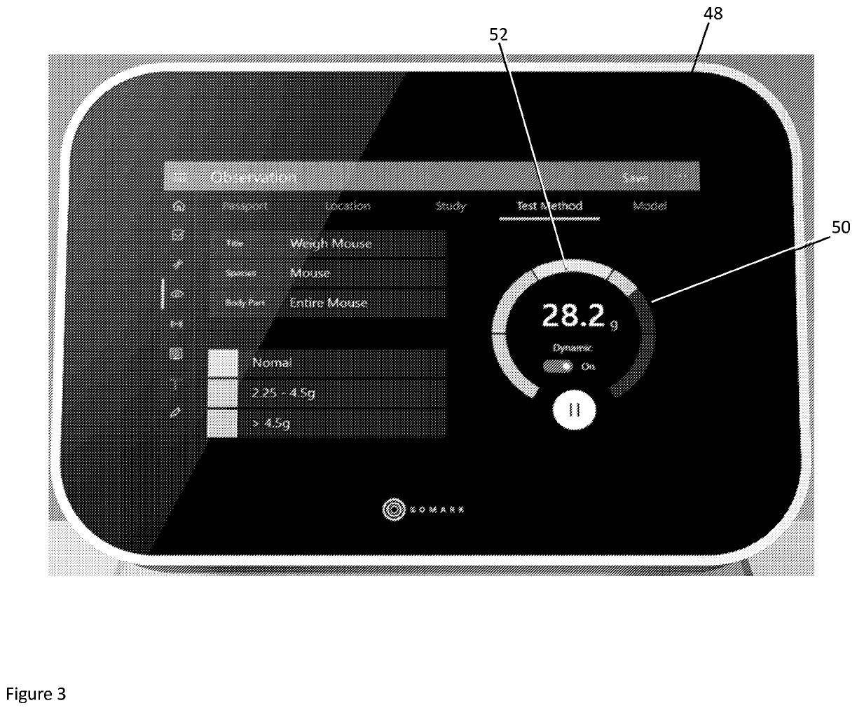

The processor 34 receives user instruction information indicative of an instruction for a user in the form of an animal technician to initiate the generation of the empirical information relating to the laboratory animal 38. The processor communicates to the user the user the instruction. The user processor causes an electronic display 48 to display the instruction to the user. FIG. 3 is an example of a suitable electronic touchscreen display 48, which is part of a graphical user interface. Alternatively or additionally, the processor causes a speaker to voice the instruction to the user, or generally communicate the instruction in any suitable fashion. The instruction may be, for example, to weigh the animal 38 using a laboratory device in the form of scales 20 to generate empirical information in the form of animal mass information, measure the length of the animal 38 with a laboratory device in the form of callipers 22 to generate empirical information in the form of length information, determine the animals response to a stimulus using a laboratory device creating an experimental environment 24 to generate empirical information in the form of response information, and time the animals reaction time to an event using a laboratory device in the form of a timer 26 to generate reaction time information, or generally generate any empirical information relating to the laboratory animal 38, for example tumour size or compound empirical information, for example body condition score.

Generally, but not necessarily, the laboratory comprises a plurality of animals and the instructions are for the user to initiate the generation of empirical data in relation to a specified animal of the plurality of animals. The user may inadvertently select the incorrect animal. The processer confirms that the user instruction information comprises the animal identification information generated by the at least one of the plurality of animal laboratory devices, so to ensure that the right animal has engaged the at least one animal laboratory device.

The processor 34 triggers an alert when the processor determines that the empirical information satisfies an alert condition. The alert may be in the form of, for example, an audible alert, a visible alert, for example an error icon and/or error message text displayed on the display 48, or a message sent via the communications interface 30 to another machine. For example, an alert condition may be that the animal's mass has decreased by more than a percentage value. The user may be alerted if the animal is at risk or there is an error.

The laboratory equipment interface 10 comprising pre-loaded animal laboratory device software in the form of animal laboratory device drivers for each of the plurality of animal laboratory devices 16-28.

Including the animal identification and the empirical data in the same data unit may improve the integrity of the data. It is less likely that empirical data will be recorded against the wrong animal identity in the data store. The information transmission unit is a text stream in the form of a text file holding information in a data-interchange format. In the present embodiment the information transmission unit is a JSON file, which is human readable and writable. A HTTP over transport layer security (HTTPS) session between the laboratory equipment interface 10 and the server 32 is started. A POST command is used to send the information transmission unit 36 from the laboratory equipment interface 10 to the server 32. In alternative embodiments, the information transmission unit 10 comprises a XML file, a CSV file, or generally be any suitable information transmission unit. FTP or any suitable service may be used instead of HTTPS.

The laboratory equipment interface has a network address in the form of a IEEE 802.3 MAC address and an internet protocol address, and the processor includes the network address in the information transmission unit. The network address is included by the processor 34 in the information transmission unit 36 and associated in the data store with the empirical information. This enables tracking of the source of the empirical information.

The at least one communication interface comprises at least one personal area network interface. For example, communications interface 12 may be a Blue Tooth interface, communications interface 14 may be a universal serial bus (USB) interface, and communications interface 16 may be a RS-232 interface. One of the plurality of communication interfaces may be, however, a different type of interface, for example an IEEE 802.11 (“Wi-Fi”) communications interface, a local area network interface in the form of an IEEE 802.3 (“Ethernet”) communications interface, or a LPWAN interface, for example Sigfox. The communications interface 30 is for a local area network that is connected to an internetwork, for example the Internet. The remote data store 32 comprises in this example a computer server running data base software connected to a respective local area network that is connected to the internet.

The laboratory equipment interface 10 comprises a housing in which the processor 34 is housed and a connected electronic touch screen display 48 that is hingedly mounted for user selection of the viewing angle. The laboratory equipment interface 10 comprises a power management system including mains to DC conversion and a battery. In the housing is a printed circuit board assembly to which is mounted non-volatile memory in the form of flash memory, random access memory and a central processing unit (CPU) in the form of a multicore intel processor. The CPU runs any suitable operating system, for example any of Windows, LINUX and IOS, for example. The non-volatile memory has factory preloaded device driver software for the plurality of animal laboratory devices, which may replace the need for the user to load device driver software. This embodiment of the laboratory equipment interface 10 includes a plurality of communication interface types, for example for a plurality of Blue Tooth Connections, multiple USB ports, and multiple RS232 ports.

An example of how a user interacts with the laboratory equipment interface 10 is now described. The user connects an identification tag reader 18 in the form of a RFID tag reader to the laboratory equipment interface 10 via a communications interface 12. The user then connects at least one animal laboratory device 24, and generally a plurality of animal laboratory devices, to one or more communications interfaces 14. The user then turns the laboratory equipment interface 10. The laboratory equipment interface 10 establishes communications with the remote data store. The user causes the identification tag reader 18 to read the RFID tag attached to an animal. The laboratory equipment interface 10 then displays at least one of the following:
The TID of the tag attached the animal
The animal identification information
Animal characteristics, examples of which include but are not limited to strain, date of birth, genotype, and other characteristics shown in FIG. 3.

The displayed information may be sent by the data store to the laboratory equipment interface 10, which is subsequently displayed.

The user then causes one of the animal laboratory devices 18-28 to generate empirical information about the animal, for example by weighing the animal or measuring a tumour size with electronic callipers, which the animal laboratory device sends to the laboratory equipment interface 10. The empirical information is displayed to the user on the electronic display. The user commands the laboratory equipment interface 10 to send the empirical information about the animal to the remote data store 32 by interacting with the user interface, in this embodiment by pressing the save button displayed on the tough screen. The laboratory equipment interface 10 generates the information transmission unit, and sends it to the remote data store via the communication interface 30 for association of the empirical information with the animal identification information.

Also shown in FIG. 3 is a user interface 50 comprising a dial divided into a plurality of arcuate bands, for example band 52. The bands correspond to empirical value ranges, in this example animal mass ranges. The band that has an empirical value range in which the empirical value falls is made prominent, in this example by increasing its brightness relative to other bands. In this example, the lowest band corresponds to a 20% weight loss band, which is indicative of the need for humane termination of the animal.

Now that embodiments have been described, it will be appreciated that embodiments may comprise at least one of the following advantages:
The integrity of the experimental data may be improved.
Human bias and error may be removed.
Less incorrectly identified and/or processed animals that may be euthanised
The user may be alerted of errors and/or causes for concern.
Clear instructions may be provided to the user.
The user may not need to load device driver software.
The source of the empirical data in the data store can be identified to track issues with the data.
Multiple communication interface types may be supported.

Variations and/or modifications may be made to the embodiments described without departing from the spirit or ambit of the invention. The present embodiments are, therefore, to be considered in all respects as illustrative and not restrictive. Reference to a feature disclosed herein does not mean that all embodiments must include the feature.

Prior art, if any, described herein is not to be taken as an admission that the prior art forms part of the common general knowledge in any jurisdiction.

In the claims which follow and in the preceding description of the invention, except where the context requires otherwise due to express language or necessary implication, the word “comprise” or variations such as “comprises” or “comprising” is used in an inclusive sense, that is to specify the presence of the stated features but not to preclude the presence or addition of further features in various embodiments of the invention.

1. A laboratory equipment interface comprising:
at least one communication interface for receiving information from an animal laboratory device;
a communications interface for communicating with a remote data store; and
a processor configured to generate an information transmission unit comprising empirical information relating to a laboratory animal received from a plurality of animal laboratory device and animal identification information indicative of the identity of the animal, and configured to transmit the information transmission unit to the remote data store via the communication interface for association of the empirical information with the animal identification information within the remote data store.

2. A laboratory equipment interface defined by claim 1 wherein the processor is configured to receive the animal identification information.

3. A laboratory equipment interface defined by claim 2 wherein the animal identification information is received from one of the plurality of animal laboratory devices configured to read an identification tag.

4. A laboratory animal defined by claim 2 wherein the animal identification information is received from the remote data store.

5. A laboratory equipment interface defined by claim 2, wherein the processor is configured to receive user instruction information indicative of an instruction for a user to initiate the generation of the empirical information relating to the laboratory animal and communicate to the user the instruction, and subsequently confirm that the user instruction information comprises animal identification information indicative of the animal.

6. A laboratory equipment interface defined by claim 5 wherein the processor is configured to trigger an alert when the processor determines that the empirical information satisfies an alert condition.

7. A laboratory equipment interface defined by claim 1 comprising pre-loaded animal laboratory device software for each of the plurality of animal laboratory devices.

8. (canceled)

9. (canceled)

10. A laboratory equipment interface defined by claim 1 wherein the at least one communication interface comprises at least one personal area network interface.

11. (canceled)

12. A method of associating within a data store empirical information for a laboratory animal from a plurality of animal laboratory devices, the method comprising the steps of:
receiving empirical information for a laboratory animal from each of a plurality of animal laboratory devices via at least one communications interface;
generating an information transmission unit for each of the plurality of animal laboratory devices, the information transmission unit comprising the empirical information received therefrom and animal identification information indicative of the identity of the animal; and
sending, via a communications interface for communication with a remote data store, the information transmission unit generated for each of the plurality of connected animal laboratory devices to the remote data store for association of the empirical information with the animal identity information within the remote data store.

13. A method defined by claim 12 comprising the step of receiving animal identification information.

14. A method defined by claim 13 wherein the animal identification information is received from one of the plurality of animal laboratory devices configured to read an identification tag attached to the animal.

15. A method defined by claim 12 wherein the animal identification information is received from the remote data store.

16. A method defined by claim 13 comprising the step of receiving user instruction information indicating that a user should initiate the generation of the empirical information relating to the laboratory animal and communicate to the user the user instruction information, and subsequently confirming that the user instruction information comprises animal identification information indicative of the animal.

17. A method defined by claim 16 comprising the step of triggering an alert when the processor determines that the empirical information satisfies an alert condition.

18. A method defined by claim 12 comprising the step of preloading animal laboratory device software for each of the plurality of animal laboratory devices.

19. (canceled)

20. (canceled)

21. (canceled)

22. (canceled)

23. (canceled)

24. A laboratory equipment interface comprising:
at least one communication interface for receiving information from an animal laboratory device;
a communications interface for communicating with a remote data store; and
a processor and tangible media including program instructions which when executed by the processor causes the processor to generate an information transmission unit comprising empirical information relating to a laboratory animal received from at one of a plurality of animal laboratory devices and animal identification information indicative of the identity of the animal, and transmit the information transmission unit to the remote data store via the communication interface for association of the empirical information with the animal identification information within the remote data store.
